(19) United States
(12) Patent Application Publication
(10) Pub. No.: US 2025/0276974 A1
(43) Pub. Date: Sep. 4, 2025

(54) PROCESS FOR PREPARING ZANUBRUTINIB IN AMORPHOUS FORM

(71) Applicant: OLON S.P.A., Rodano (MI) (IT)

(72) Inventors: Jacopo BONANOMI, Rodano (MI) (IT); Jacopo LESMA, Rodano (MI) (IT); Barbara NOVO, Rodano (MI) (IT); Simona TRABACE, Rodano (MI) (IT)

(73) Assignee: OLON S.P.A., Rodano (MI) (IT)

(21) Appl. No.: 18/863,759

(22) PCT Filed: May 11, 2023
(86) PCT No.: PCT/IB2023/054862

§ 371 Date: Nov. 7, 2024

(30) Foreign Application Priority Data
May 12, 2022 (IT) 102022000009872

(51) Int. Cl. C07D 487/04; C07C 65/03; C07C 229/60

(52) U.S. Cl. CPC C07D 487/04; C07C 65/03; C07C 229/60; C07B 2200/13

(57) ABSTRACT

The present invention relates to a Process for preparing Zanubrutinib in amorphous form that provides for the use of a specific co-crystal of zanubrut inib as an intermediate. The present invention also relates to zanubrutinib co-crystals, which can be used in this process. The present invention also relates to a process for preparing said Zanubrutinib co-crystals.

### TECHNICAL FIELD

The present invention relates to a Process for preparing Zanubrutinib in amorphous form, which provides for the use of a specific co-crystal of zanubrutinib as an intermediate. The present invention also relates to zanubrutinib co-crystals that can be used in this process. The present invention also relates to a process for preparing said Zanubrutinib co-crystals.

### PRIOR ART

Zanubrutinib is an active ingredient classified as a Bruton Tyrosine Kinase (BTK) Inhibitor, currently approved by the FDA for the treatment of mantle cell lymphoma (MCL) in patients who have received at least one prior therapy. It is marketed under the name Brukinsa®. WO 2014/173289, in the name of Beigene LTD, describes Zanubrutinib and its preparation (compound 27).

The synthesis process of zanubrutinib is also described in WO 2018/033135. In the same patent, in particular in example 1, paragraph [0114], the purification of zanubrutinib by column chromatography is described. However, this process has a complex scale-up and long execution times. Therefore, this technique is difficult to industrialize.

WO 2018/033853 discloses a crystalline form (form A) of zanubrutinib, the amorphous form and related preparation processes. Form A is further mentioned as a result of example 1, step 16 in WO 2019/108795. Also in this patent, the purification of the product is carried out by means of a chromatographic column, since form A is not easily crystallizable and therefore purifiable by crystallization.

Patent WO 2021/259732, in the name of Sandoz AG, describes two specific co-crystals of zanubrutinib, wherein the coformers are 4-hydroxybenzoic acid and 3,4-dihydroxybenzoic acid, respectively.

Therefore, the need is felt to find a process for producing zanubrutinib which is easy to industrialize and which provides for acceptable execution times for production on an industrial scale.

### SUMMARY OF THE INVENTION

The Applicant has now found a Process for preparing Zanubrutinib in amorphous form which solves said problems, providing for the path through a co-crystal selected from those defined below, which allow a simpler scale-up of the process with respect to the prior art, since such co-crystals can be purified by crystallization, without the use of chromatographic columns.

Therefore, according to a first aspect, the present invention relates to a process for preparing zanubrutinib in amorphous form according to the attached claims.

According to another aspect, the present invention relates to zanubrutinib co-crystals according to the attached claims.

According to another aspect, the present invention relates to a process for preparing the aforementioned Zanubrutinib co-crystals according to the attached claims.

Advantageously, the co-crystals of the present invention, used as intermediates in the preparation of amorphous zanubrutinib, allow a simpler scale-up compared to the techniques used in the known art, for example the chromatographic techniques, as well as a reduction in execution times. Furthermore, the process according to the present invention is characterized by a very reliable repeatability, such as to allow an advantageous re-processing of any product, which does not comply with the release specifications. This process also allows obtaining a product characterized by a high purity.

### BRIEF DESCRIPTION OF THE FIGURES

FIG. 1 shows the hydrogen nuclear magnetic resonance (¹H-NMR) spectrum of the co-crystal Zanubrutinib with 3-hydroxybenzoic acid.

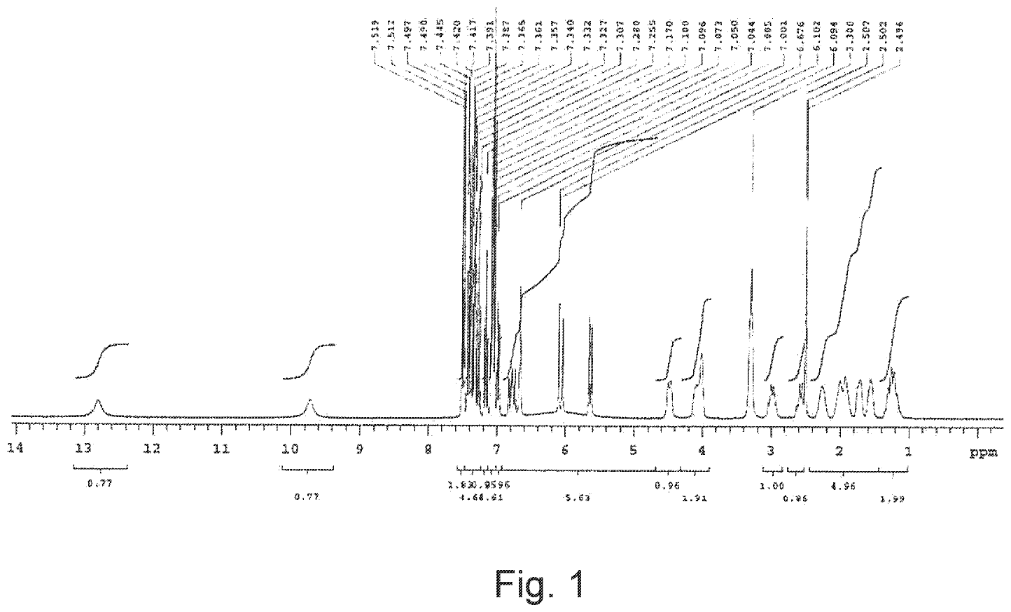

FIG. 2 shows the infrared (IR) spectrum of the co-crystal of zanubrutinib with 3-hydroxybenzoic acid.

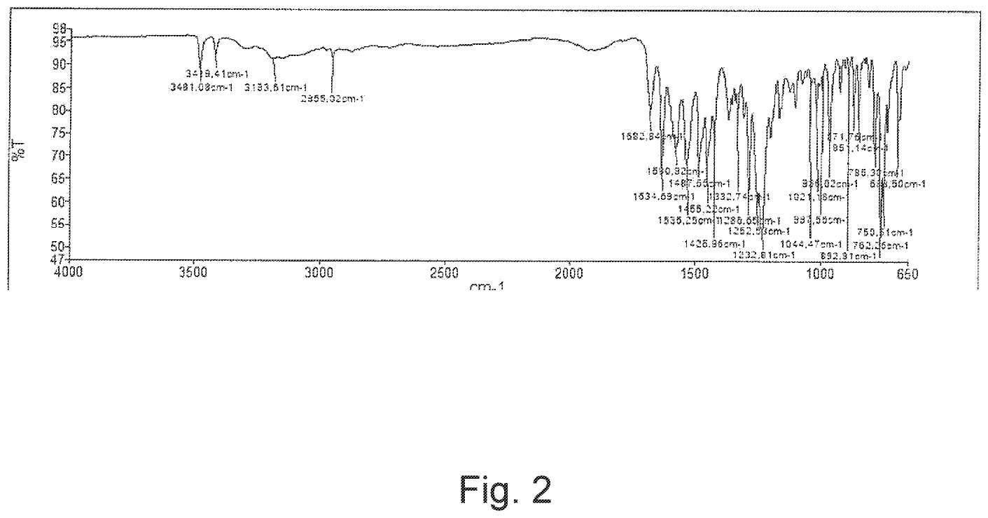

FIG. 3 shows the plot of the differential scanning calorimetry (DSC) analysis of the co-crystal zanubrutinib with 3-hydroxybenzoic acid.

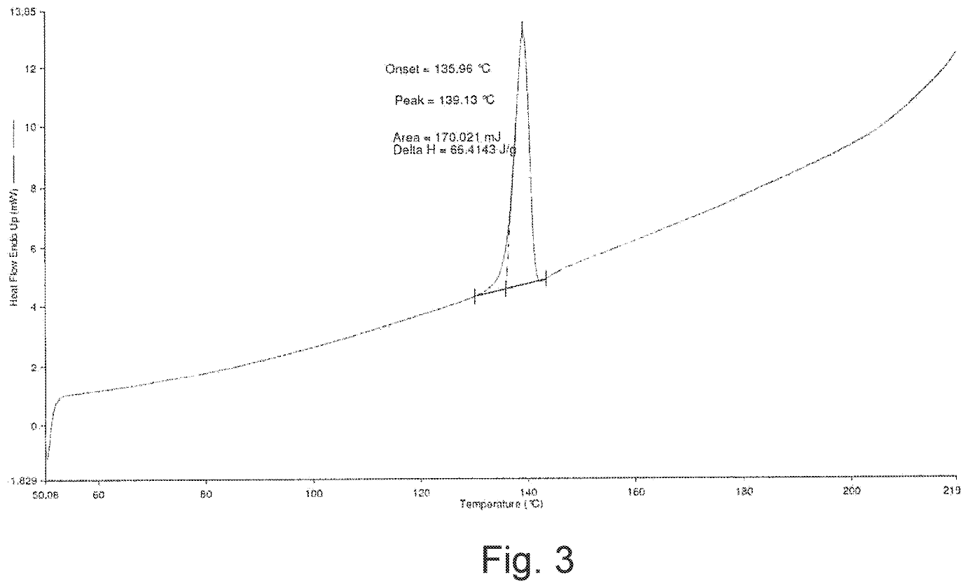

FIG. 4 shows the X-ray diffraction (XRPD) spectrum of the Zanubrutinib co-crystal with 3-hydroxybenzoic acid.

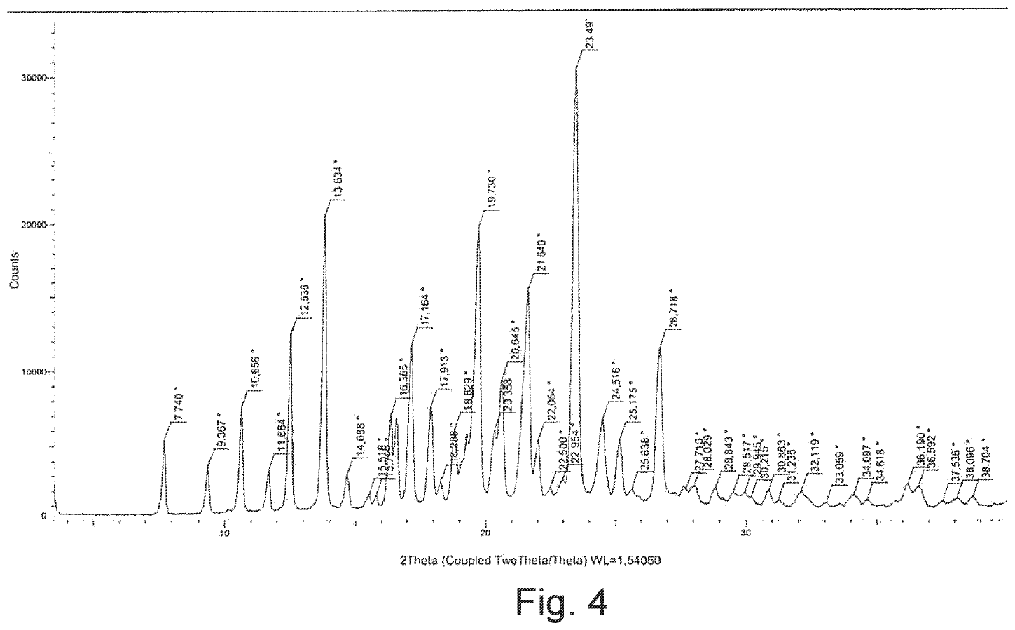

FIG. 5 shows the thermogravimetric analysis (TGA) plots of the co-crystal Zanubrutinib with 3-hydroxybenzoic acid.

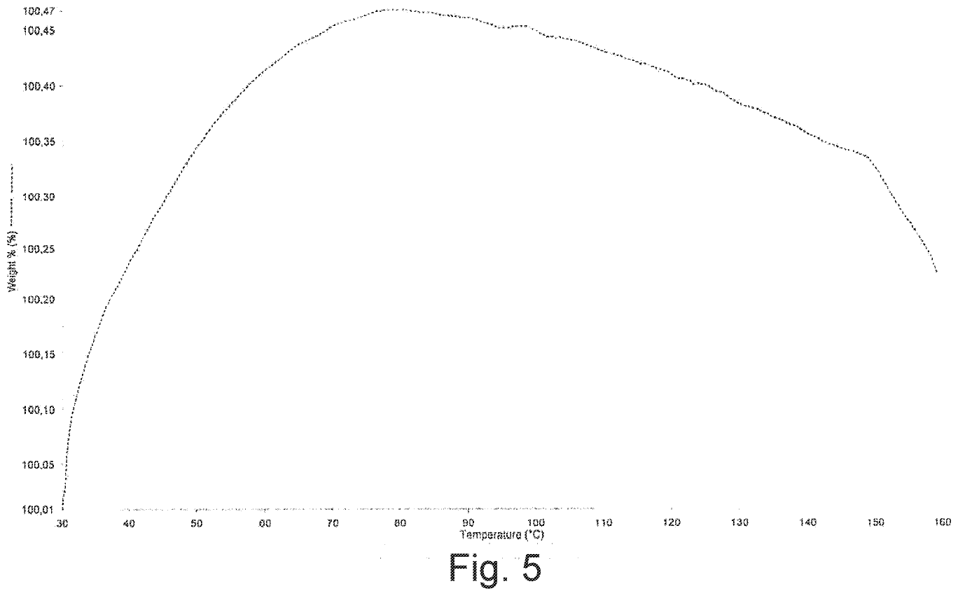

FIG. 6 shows the hydrogen nuclear magnetic resonance (¹H-NMR) spectrum of the co-crystal Zanubrutinib with 2,4-dihydroxybenzoic acid.

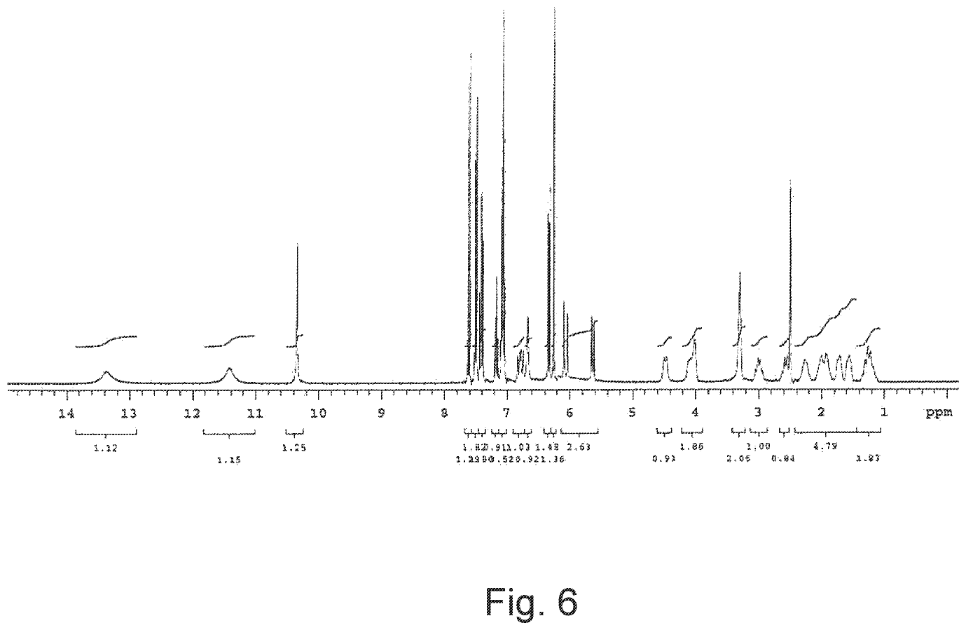

FIG. 7 shows the infrared spectrum (IR) of the co-crystal of zanubrutinib with 2,4-dihydroxybenzoic acid.

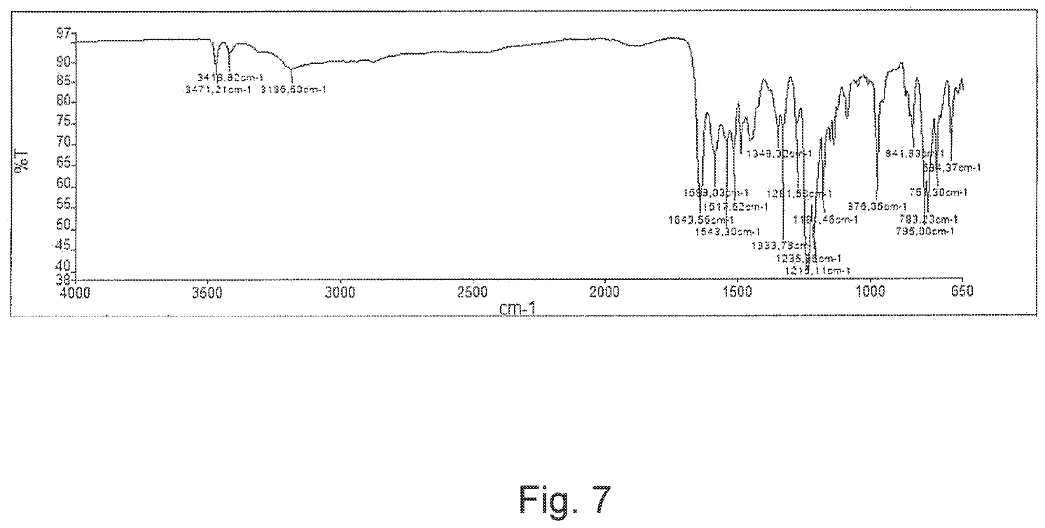

FIG. 8 shows the differential scanning calorimetry (DSC) analysis plot of the co-crystal Zanubrutinib with 2,4-dihydroxybenzoic acid.

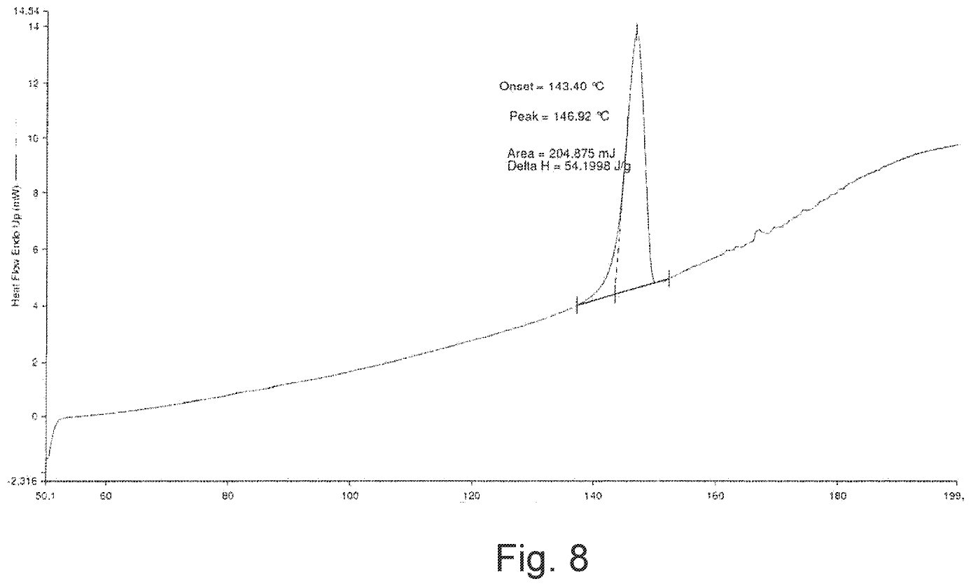

FIG. 9 shows the X-ray diffraction (XRPD) spectrum of the co-crystal Zanubrutinib with 2,4-dihydroxybenzoic acid.

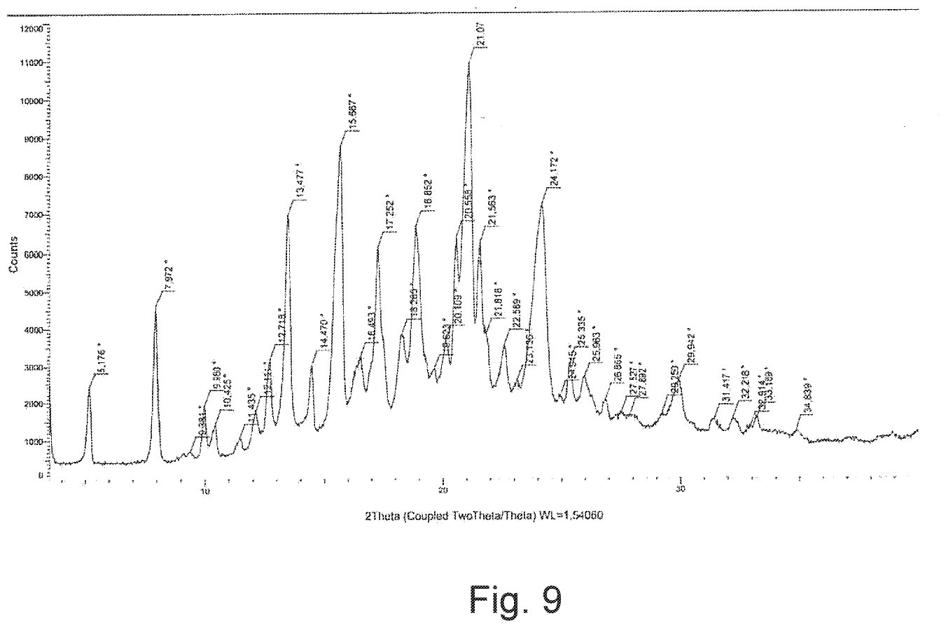

FIG. 10 shows the thermogravimetric analysis (TGA) plots of the co-crystal Zanubrutinib with 2,4-dihydroxybenzoic acid.

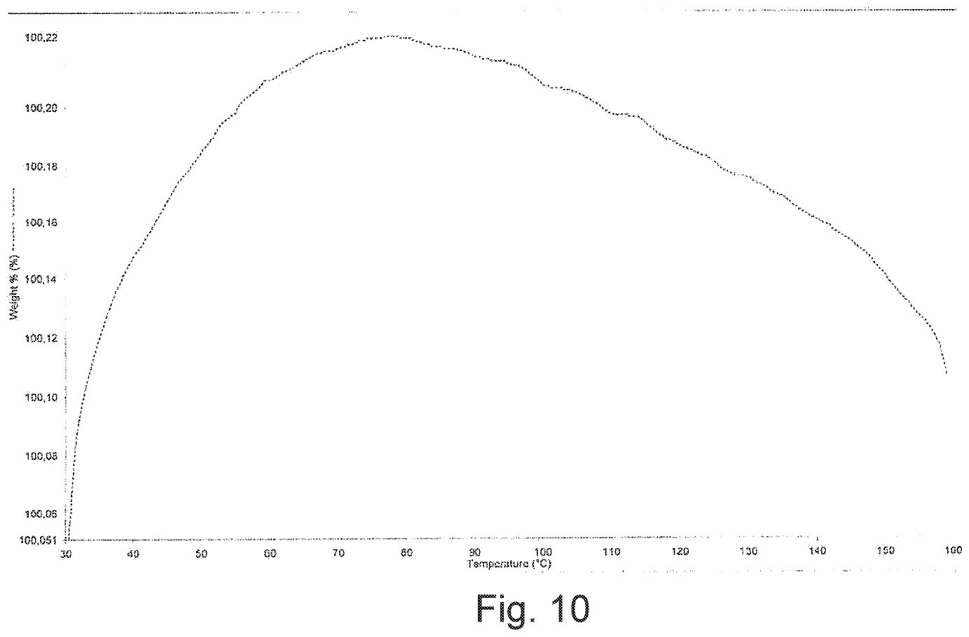

FIG. 11 shows the hydrogen nuclear magnetic resonance (¹H-NMR) spectrum of the co-crystal Zanubrutinib with 4-aminobenzoic acid.

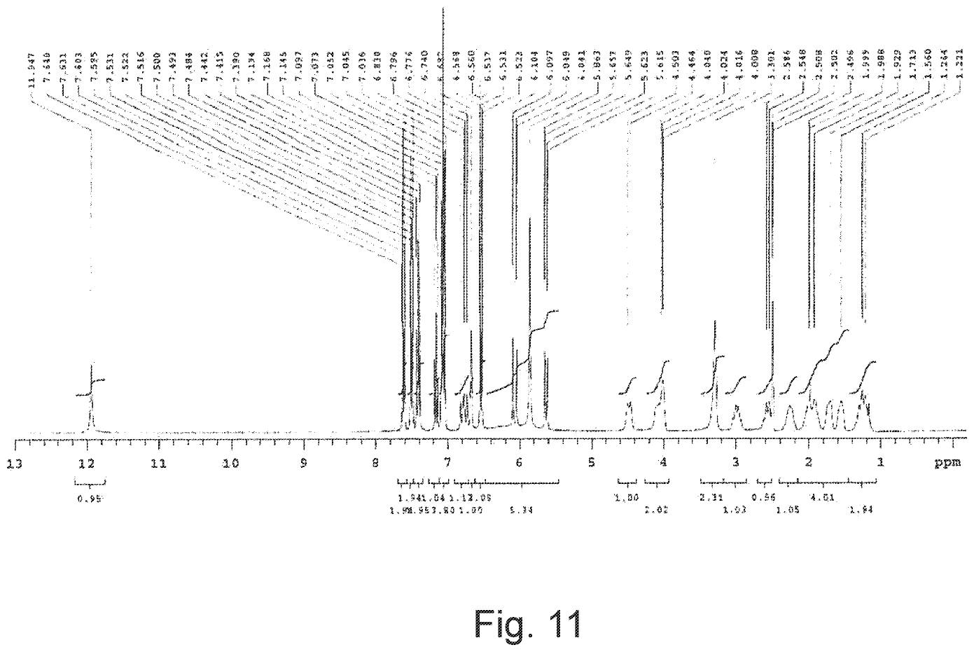

FIG. 12 shows the infrared (IR) spectrum of the co-crystal of zanubrutinib with 4-aminobenzoic acid.

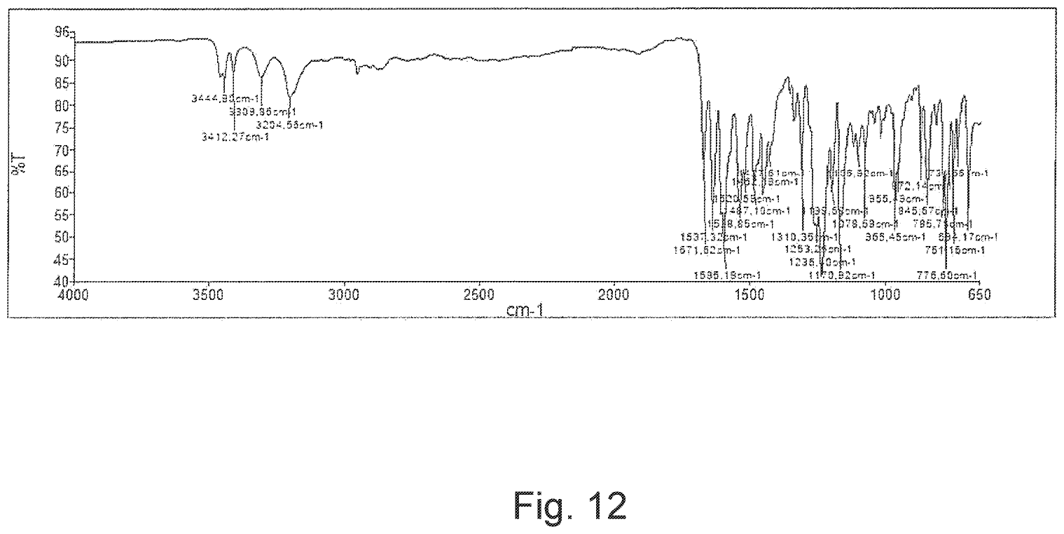

FIG. 13 shows the differential scanning calorimetry (DSC) analysis plot of the co-crystal Zanubrutinib with 4-aminobenzoic acid.

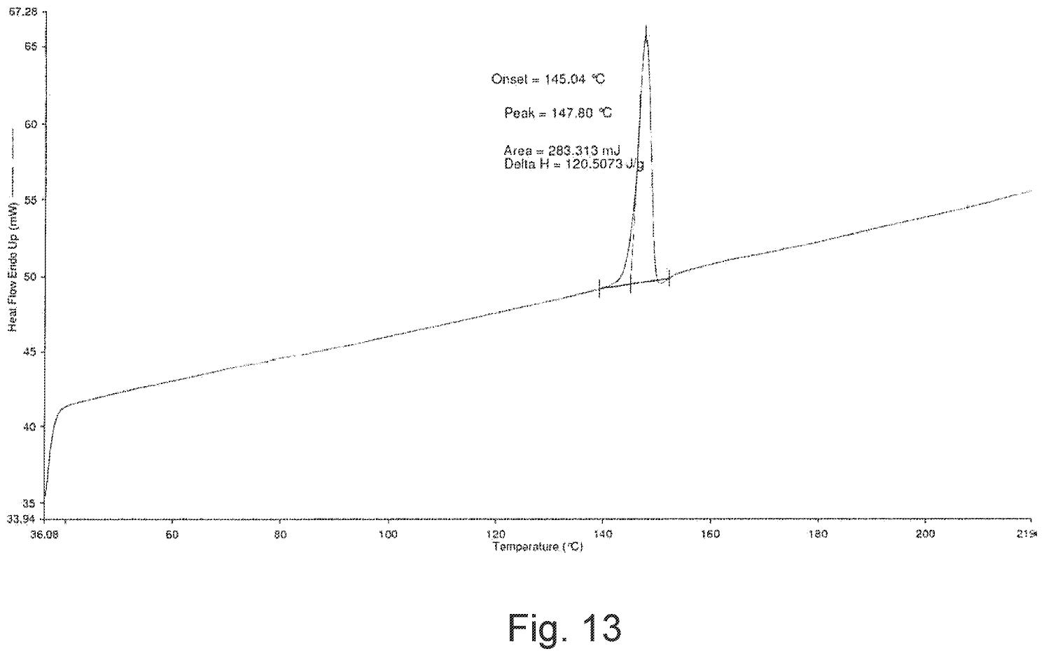

FIG. 14 shows the X-ray diffraction (XRPD) spectrum of the co-crystal Zanubrutinib with 4-aminobenzoic acid.

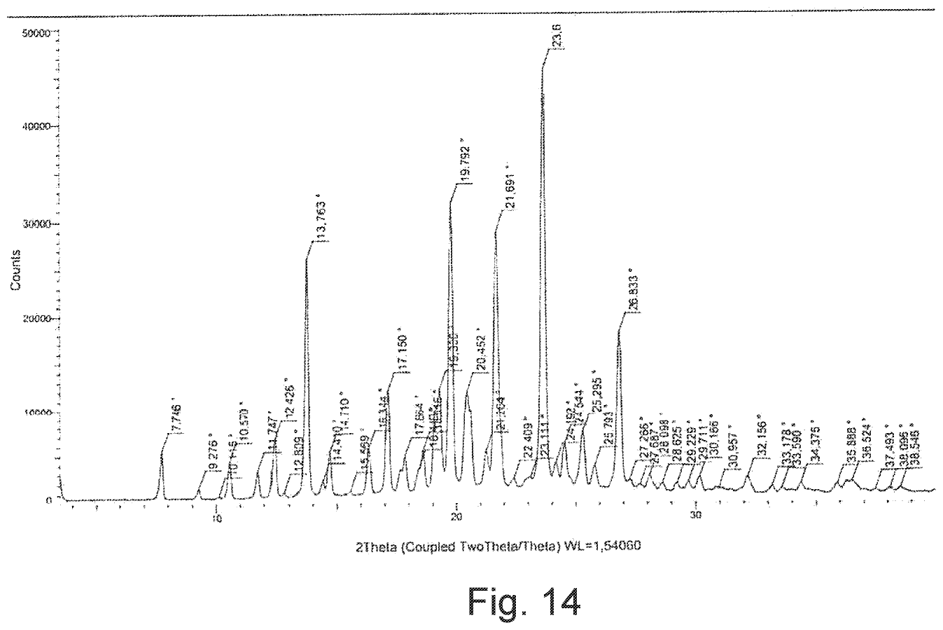

FIG. 15 shows the thermogravimetric analysis (TGA) plots of the co-crystal Zanubrutinib with 4-aminobenzoic acid.

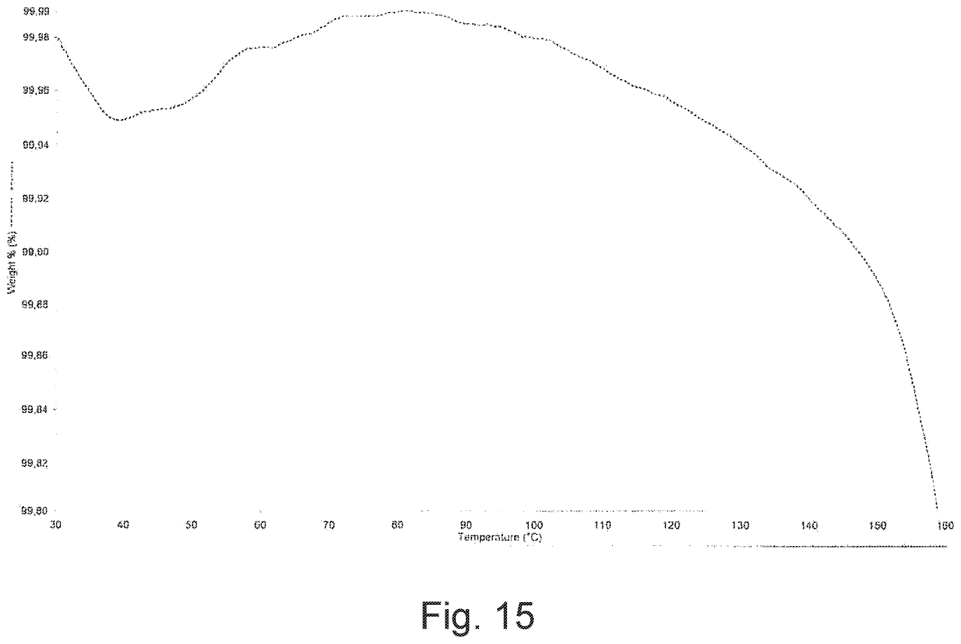

FIG. 16 shows the hydrogen nuclear magnetic resonance (¹H-NMR) spectrum of the zanubrutinib co-crystal with 4-methylaminobenzoic acid.

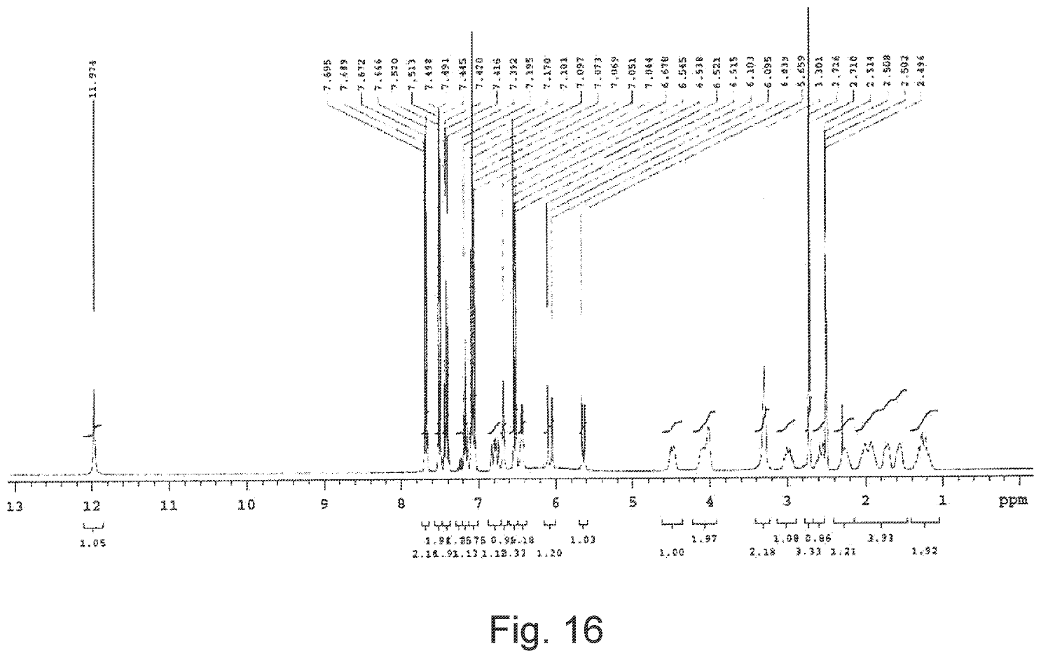

FIG. 17 shows the infrared spectrum (IR) of the co-crystal of zanubrutinib with 4-methylaminobenzoic acid.

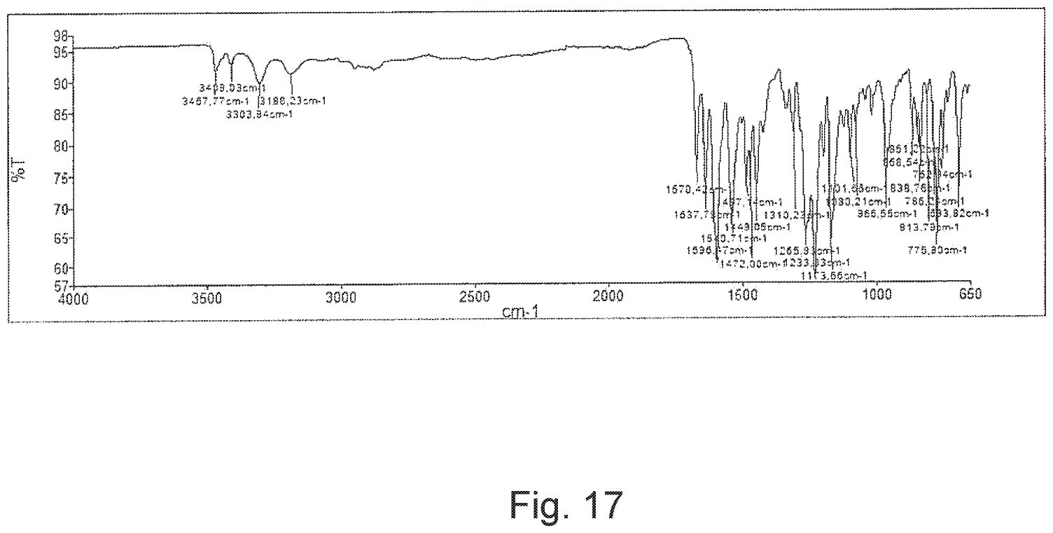

FIG. 18 shows the differential scanning calorimetry (DSC) analysis plot of the co-crystal Zanubrutinib with 4-methylaminobenzoic acid.

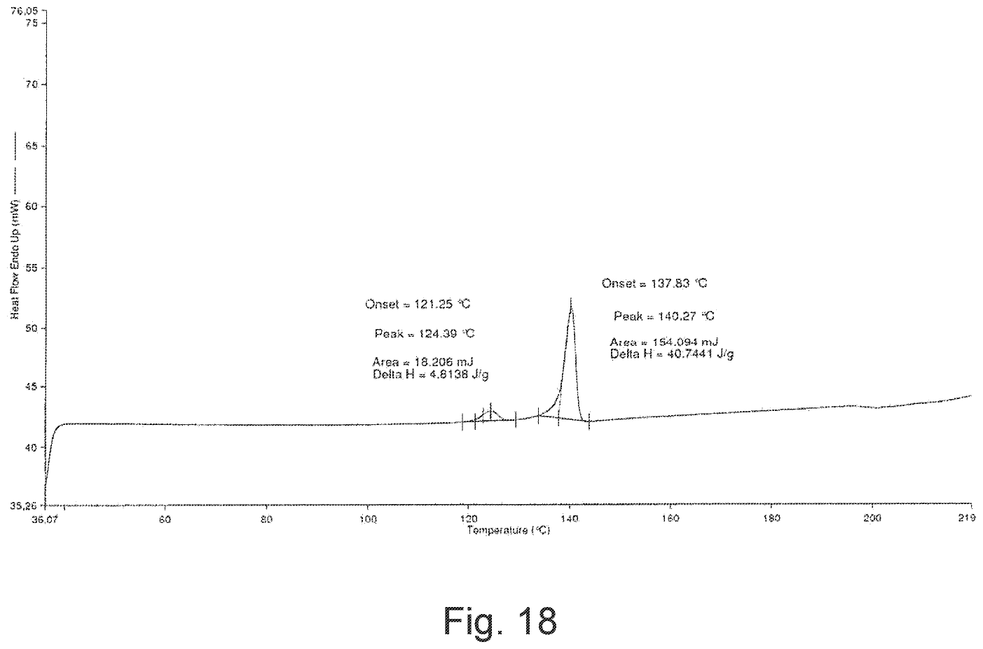

FIG. 19 shows the X-ray diffraction (XRPD) spectrum of the Zanubrutinib co-crystal with 4-methylaminobenzoic acid.

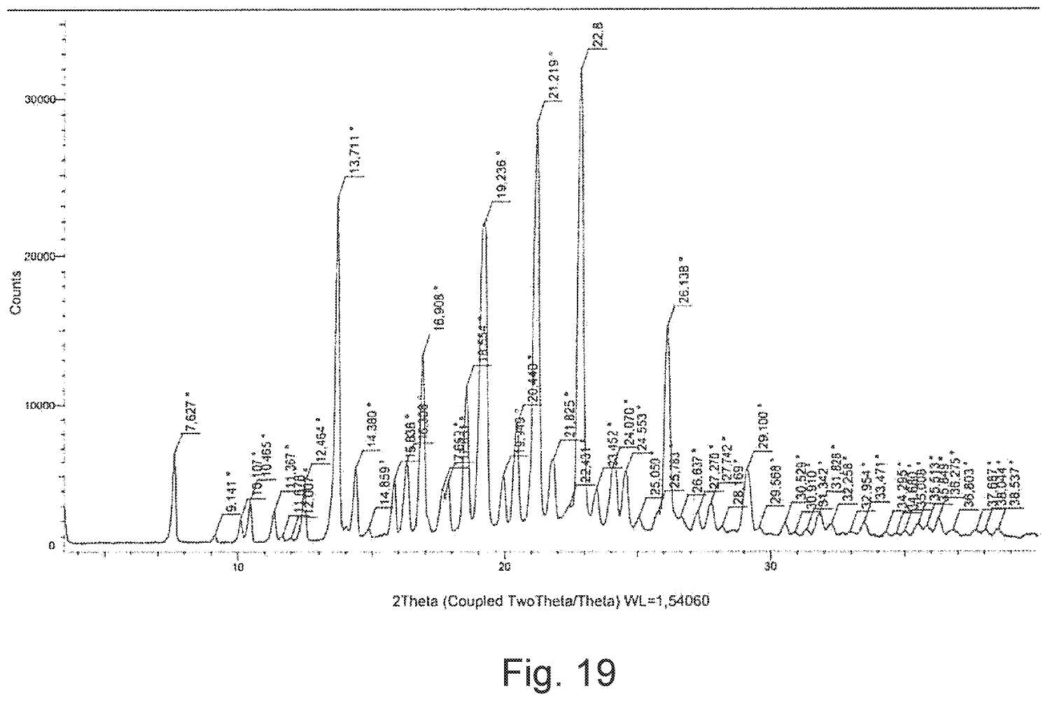

FIG. 20 shows the thermogravimetric analysis (TGA) plots of the co-crystal Zanubrutinib with 4-methylaminobenzoic acid.

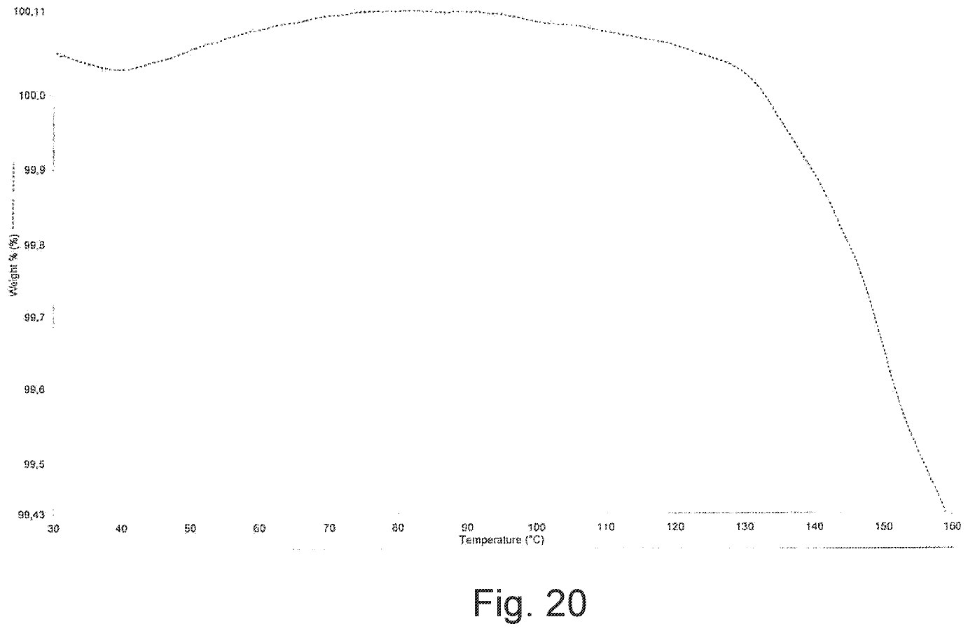

FIG. 21 shows the X-ray diffraction (XRPD) spectrum of amorphous zanubrutinib obtained from Example 2.

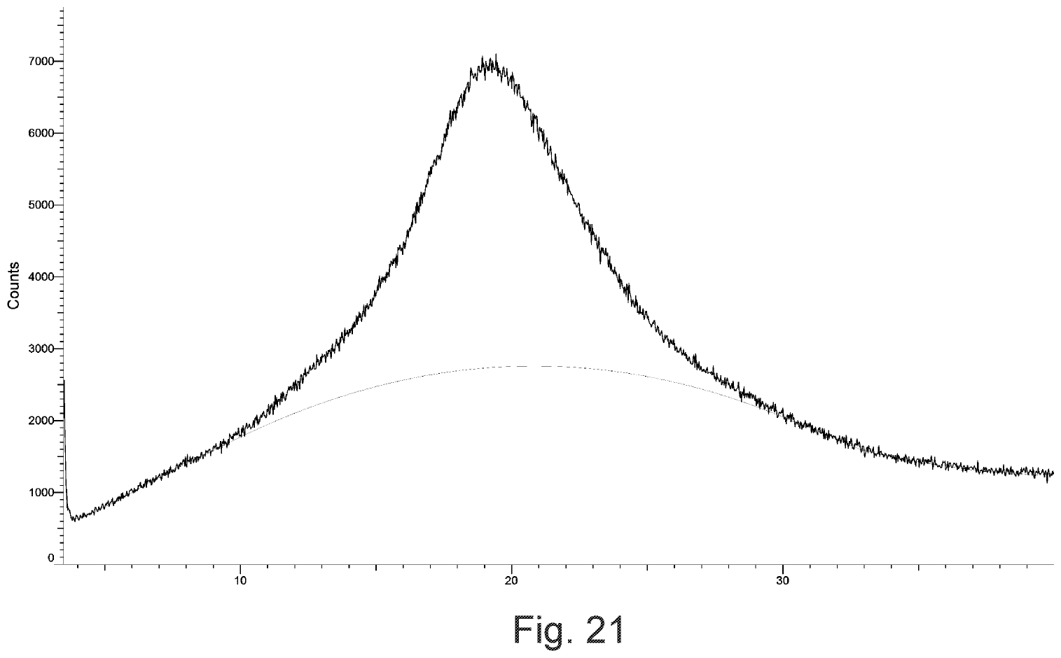

### DETAILED DESCRIPTION OF THE INVENTION

For the purposes of the invention, definitions of some terms and/or expressions used in the present description and in the claims will be provided below.

A first object of the invention therefore relates to a process for preparing zanubrutinib of formula (I)

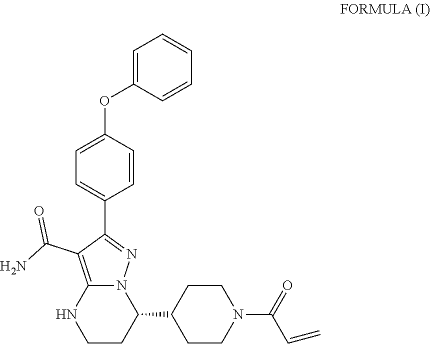 [SMILES: C=CC(=O)N1CCC([C@@H]2CCNc3c(C(N)=O)c(-c4ccc(Oc5ccccc5)cc4)nn32)CC1]

in amorphous form, comprising the steps of:
a) reacting a compound of formula (II), or a salt thereof:

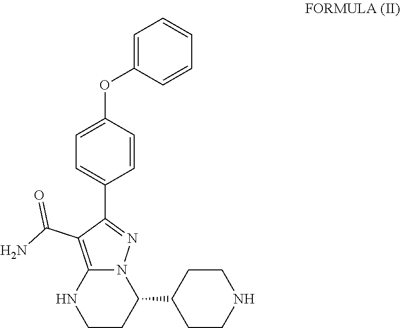 [SMILES: NC(=O)c1c(-c2ccc(Oc3ccccc3)cc2)nn2c1NCC[C@H]2C1CCNCC1]

with acryloyl chloride to form zanubrutinib;
b) adding to Zanubrutinib thus obtained a coformer selected from: 3-hydroxybenzoic acid, 2,4-dihydroxybenzoic acid, 4-aminobenzoic acid and 4-methylaminobenzoic acid, to obtain the corresponding Zanubrutinib co-crystal;
c) converting zanubrutinib co-crystal obtained in step b) to zanubrutinib in amorphous form.

Step a) of the process of the invention provides for the reaction of a compound of formula (II) or a salt thereof. Said salt is preferably selected from the salts of the compound of formula (II) with an acid selected from: L-DBTA (L-dibenzoyltartaric acid), D-DBTA (D-dibenzoyltartaric acid), L-DTTA (L-dibenzoyltartaric p-toluyl-tartaric), D-DTTA (D-di-p-toluyl-tartaric acid), L-malic acid, D-malic acid, L-mandelic acid, D-mandelic acid, L-camphorsulfonic acid, D-acid-camphorsulphonic, L-tartaric acid, D-tartaric acid. Preferably the salt is a salt of the compound of formula (II) with L-dibenzoyltartaric or D-dibenzoyltartaric acid, more preferably L-dibenzoyltartaric acid.

As reported above, the coformer of step b) is selected from: 3-hydroxybenzoic acid, 2,4-dihydroxybenzoic acid, 4-aminobenzoic acid and 4-methylaminobenzoic acid.

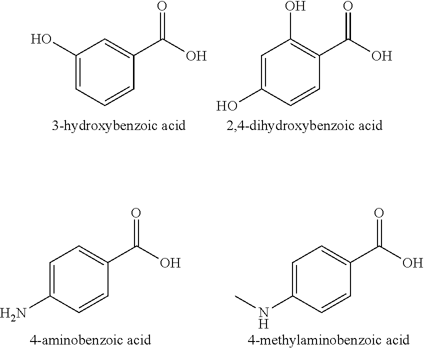

From step b) Zanubrutinib co-crystal is obtained. This co-crystal can advantageously be purified by providing for one or more recrystallization processes.

Preferably, in the Zanubrutinib co-crystal the molar ratio of Zanubrutinib to the coformer is 1:0.95 to 1:1.05.

According to a preferred aspect, this co-crystal has a purity equal to or higher than 98.5%, preferably equal to or higher than 99.0%, even more preferably equal to or higher than 99.5%.

Advantageously, the process is characterized by a high capacity to remove any impurities present in the zanubrutinib. In particular, the process of the invention allows removing and therefore controlling the levels of the following impurities:

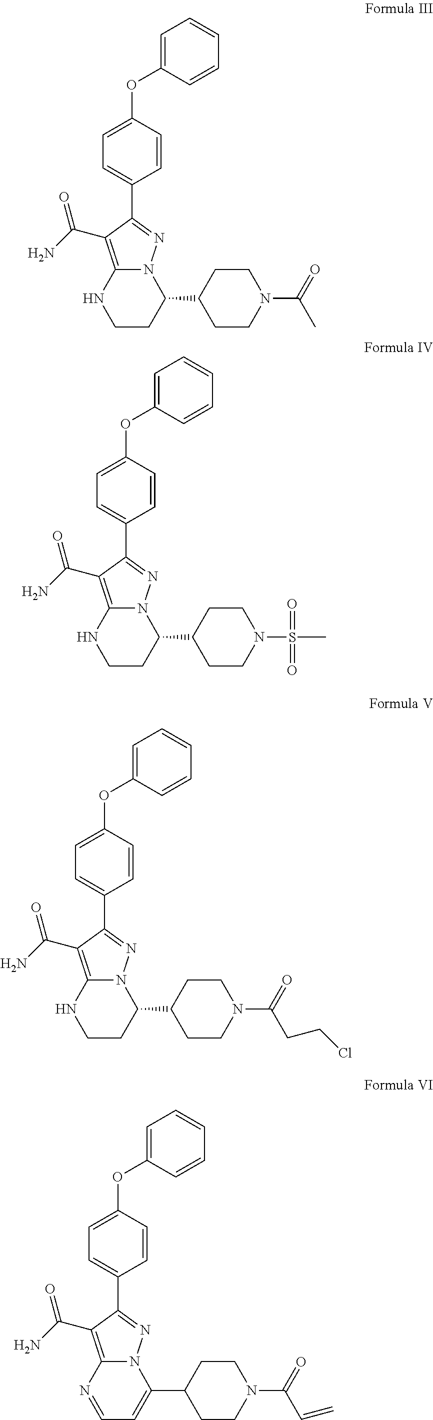

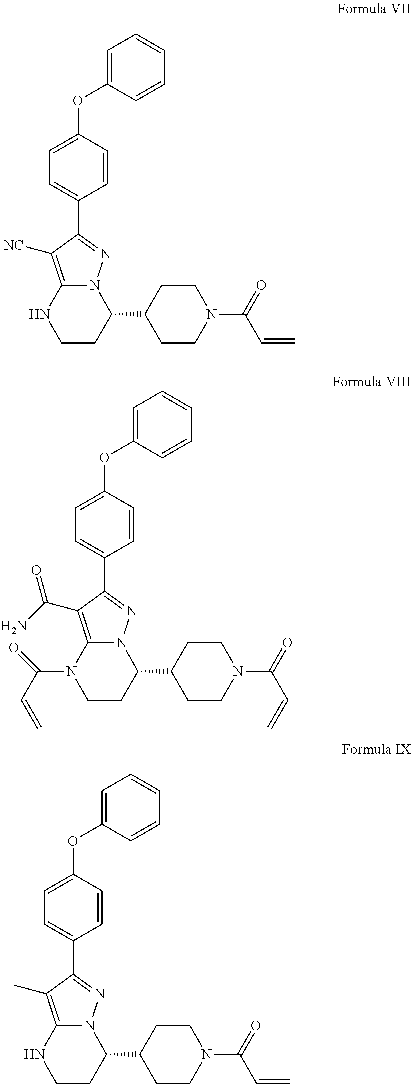

According to a preferred aspect, in particular when a salt is reacted in step a), step a) of the process of the invention is carried out under basic conditions, preferably by adding sodium hydroxide (NaOH) or sodium bicarbonate. Preferably the pH is greater than or equal to 9. Preferably the pH is from 9 to 12.

According to a particularly preferred aspect, NaOH is used, preferably NaOH 30% aq.

The following Scheme 1 shows the process of the invention according to a particular preferred embodiment. Specifically, scheme 1 refers to a particular embodiment wherein step a) provides for the use of the co-crystal of Zanubrutinib with L-dibenzoyltartaric acid (L-DBTA):

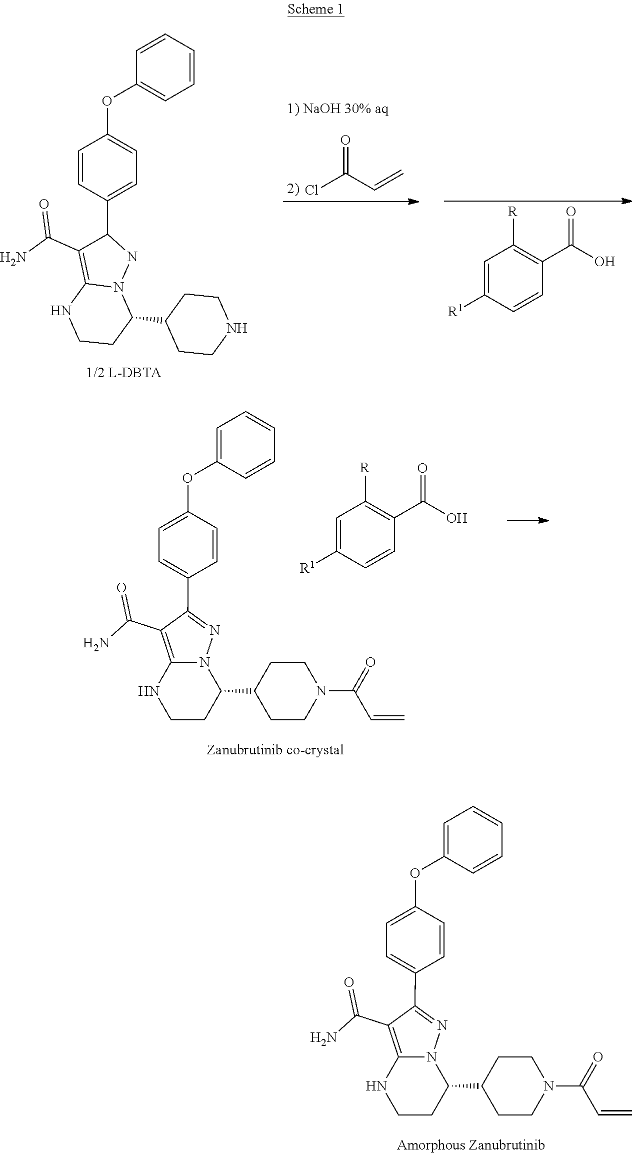

According to another preferred aspect, the conversion step c) takes place by treating the co-crystal in water at controlled pH, preferably at basic pH. Preferably, the pH is greater than or equal to 9. Preferably, the pH is from 9 to 12. The conversion step c) allows the precipitation of amorphous zanubrutinib. The conversion step c) allows to remove the salified coformer.

According to a preferred aspect, when the coformer of the co-crystal is 3-hydroxybenzoic acid, the co-crystal is characterized by an XRPD diffractogram comprising at least one reflection at angle 2θ selected from: 12.5°±0.2°, 13, 8°±0.2°, 17.2°±0.2°, 19.7°∓0.2°, 21.6°±0.2°, 23.5°±0.2°, 26, 7°±0.2°. Preferably, said XRPD diffractogram comprises at least one reflection at an angle 2θ selected from: 13.8°±0.2°, 19.7°±0.2°, 23.5°±0.2°. According to another preferred aspect, the co-crystal is characterized by an XRPD diffractogram comprising a reflection at angle 2θ equal to 23.5°±0.2°. According to a further particularly preferred aspect, said XRPD diffractogram comprises reflections at angles 2θ equal to 13.8°±0.2°, 19.7°±0.2°, 23.5°±0.2°.

In a preferred embodiment, when the coformer of the co-crystal is 3-hydroxybenzoic acid, the co-crystal is characterized by an XRPD diffractogram comprising a reflection angle 2θ equal to 23.5°±0.2°. Preferably, said co-crystal further comprises at least one reflection at an angle 2θ selected from: 12.5°±0.2°, 13.8°±0.2°, 17.2°±0.2°, 19.7°±0.2°, 21.6°±0.2°, 26.7°±0.2°, preferably 13.8°±0.2° and 19.7°±0.2°.

According to a preferred aspect, when the coformer of the co-crystal is 2,4-dihydroxybenzoic acid, the co-crystal is characterized by an XRPD diffractogram comprising at least one reflection at angle 2θ selected from: 5.2°±0.2°, 7.9°±0.2°, 13.5°±0.2°, 15.7°±0.2°, 17.2°±0.2°, 18.8°±0.2°, 21.1°±0.2°, 24.2°±0.2°. Preferably, said XRPD diffractogram comprises at least one reflection at an angle 2θ selected from: 13.5°±0.2°, 15.7°±0.2°, 21.1°±0.2°. According to another preferred aspect, the co-crystal is characterized by an XRPD diffractogram comprising a reflection at angle 2θ equal to 21.1°±0.2°. According to a further particularly preferred aspect, said XRPD diffractogram comprises reflections at angles 2θ equal to 13.5°±0.2°, 15.7°±0.2°, 21.1°±0.2°.

In a preferred embodiment, when the coformer of the co-crystal is 2,4-dihydroxybenzoic acid, the co-crystal is characterized by an XRPD diffractogram comprising a reflection angle 2θ equal to 21.1°±0.2°. Preferably, said co-crystal further comprises at least one reflection at an angle 2θ selected from: 5.2°±0.2°, 7.9°±0.2°, 13.5°±0.2°, 15.7°±0.2°, 17.2°±0.2°, 18.8°±0.2°, 24.2°±0.2°, preferably 13.5°±0.2° and 15, 7°±0.2°.

According to a preferred aspect, when the coformer of the co-crystal is 4-aminobenzoic acid, the co-crystal is characterized by an XRPD diffractogram comprising at least one reflection at angle 2θ selected from: 7.7°±0.2°, 13, 8°±0.2°, 19.8°±0.2°, 21.7°±0.2°, 23.6°±0.2°, 26.8°±0.2°. Preferably, said XRPD diffractogram comprises at least one reflection at an angle 2θ selected from: 13.8°±0.2°, 19.8°±0.2°, 21.7°±0.2°, 23.6°±0.2°. According to another preferred aspect, the co-crystal is characterized by an XRPD diffractogram comprising a reflection at angle 2θ equal to 23.6°±0.2°. According to a further particularly preferred aspect said XRPD diffractogram comprises reflections at angles 2θ equal to 13.8°±0.2°, 19.8°±0.2°, 21.7°±0.2°, 23.6°±0.2°.

In a preferred embodiment, when the coformer of the co-crystal is 4-aminobenzoic acid, the co-crystal is characterized by an XRPD diffractogram comprising a reflection angle 2θ equal to 23.6°. Preferably, said co-crystal further comprises at least one reflection at an angle 2θ selected from: 7.7°±0.2°, 13.8°±0.2°, 19.8°±0.2°, 21.7°±0.2°, 26.8°±0.2°, preferably 13.8°±0.2°, 19.8°±0.2°, 21.7°±0.2°.

According to a preferred aspect, when the coformer of the co-crystal is 4-methylaminobenzoic acid, the co-crystal is characterized by an XRPD diffractogram comprising at least one reflection at angle 2θ selected from: 7.6°±0.2°, 13, 7°±0.2°, 16.9°±0.2°, 19.2°±0.2°, 21.2°±0.2°, 22.8°±0.2°, 26, 1°±0.2°. Preferably, said XRPD diffractogram comprises at least one reflection at an angle 2θ selected from: 13.7°±0.20, 16.9°±0.20, 21.2°±0.20, 22.8°±0.2°.

According to another preferred aspect, the co-crystal is characterized by an XRPD diffractogram comprising a reflection at angle 2θ equal to 22.8°±0.2°. According to a further particularly preferred aspect said XRPD diffractogram comprises reflections at angles 2θ equal to 13.7°±0.2°, 16.9°±0.2°, 21.2°±0.2°, 22.8°±0.2°.

In a preferred embodiment, when the coformer of the co-crystal is 4-aminobenzoic acid, the co-crystal is characterized by an XRPD diffractogram comprising a reflection angle 2θ equal to 22.8°±0.2°. Preferably, said co-crystal further comprises at least one reflection at an angle 2θ selected from: 7.6°±0.2°, 13.7°±0.2°, 16.9°±0.2°, 19.2°±0.2°, 21.2°±0.2°, 26.1°±0.2°, preferably 13.7°±0.2°, 16.9°±0.2°, 21, 2°±0.2°.

According to a preferred aspect, when the coformer of the co-crystal is 3-hydroxybenzoic acid, the co-crystal exhibits an onset peak in DSC at a temperature between 133.9° C. and 139.9° C., preferably between 134.9° C. ° C. and 138.9° C.

According to a preferred aspect, when the coformer of the co-crystal is 2,4-dihydroxybenzoic acid, the co-crystal exhibits an onset peak in DSC at a temperature between 138.3° C. and 144.3° C., preferably between 139.3° C. and 143.3° C.

According to a preferred aspect, when the coformer of the co-crystal is 4-aminobenzoic acid, the co-crystal exhibits an onset peak in DSC at a temperature between 141.5° C. and 147.5° C., preferably between 142.5° C. ° C. and 146.5° C.

According to a preferred aspect, when the coformer of the co-crystal is 4-methylaminobenzoic acid, the co-crystal exhibits an onset peak in DSC at a temperature between 136.9° C. and 142.9° C., preferably between 137.9° C. ° C. and 141.9° C.

According to a preferred aspect, when the coformer of the co-crystal is 3-hydroxybenzoic acid, the co-crystal is characterized by an XRPD diffractogram comprising a reflection at angle 2θ equal to 23.5°±0.2° or an onset peak in DSC at a temperature between 133.9° C. and 139.9° C., preferably between 134.9° C. and 138.9° C. Preferably, said co-crystal further comprises at least one reflection at an angle 2θ selected from: 12.5°±0.2°, 13.8°±0.2°, 17.2°±0.2°, 19.7°±0.2°, 21.6°±0.2°, 26.7°±0.2°, preferably 13.8°±0.2° and 19.7°±0.2°.

According to a preferred aspect, when the coformer of the co-crystal is 2,4-dihydroxybenzoic acid, the co-crystal is characterized by an XRPD diffractogram comprising a reflection at angle 2θ equal to 21.1°±0.2° or a peak of onset in DSC at a temperature between 138.3° C. and 144.3° C., preferably between 139.9° C. and 141.9° C. Preferably, said co-crystal further comprises at least one reflection at an angle 2θ selected from: 5.2°±0.2°, 7.9°±0.2°, 13.5°±0.2°, 15.7°±0.2°, 17.2°±0.2°, 18.8°±0.2°, 24.2°±0.2°, preferably 13.5°±0.2° and 15, 7°±0.2°.

According to a preferred aspect, when the coformer of the co-crystal is 4-aminobenzoic acid, the co-crystal is characterized by an XRPD diffractogram comprising a 2θ angle reflection equal to 23.6°±0.2° or an onset peak in DSC at a temperature between 141.5° C. and 147.5° C., preferably between 142.5° C. and 146.5° C. Preferably, said co-crystal further comprises at least one reflection at an angle 2θ selected from: 7.7°±0.2°, 13.8°±0.2°, 19.8°±0.2°, 21.7°±0.2°, 26.8°±0.2°, preferably 13.8°±0.2°, 19.8°±0.20, 21.70±0.2°.

According to a preferred aspect, when the coformer of the co-crystal is 4-methylaminobenzoic acid, the co-crystal is characterized by an XRPD diffractogram comprising a 2θ angle reflection equal to 22.8°±0.2° or an onset peak in DSC at a temperature between 136.9° C. and 142.9° C., preferably between 137.9° C. and 141.9° C. Preferably, said co-crystal further comprises at least one reflection at an angle 2θ selected from: 7.6°±0.2°, 13.7°±0.2°, 16.9°±0.2°, 19.2°±0.2°, 21.2°±0.2°, 26.1°±0.2°, preferably 13.7°±0.2°, 16.9°±0.2°, 21, 2°±0.2°.

In the zanubrutinib co-crystal the molar ratio between zanubrutinib and coformer is preferably from 1:0.95 to 1:1.05.

According to a preferred aspect, when the coformer of the co-crystal is 3-hydroxybenzoic acid, the molar ratio between Zanubrutinib and 3-hydroxybenzoic acid is from 1:0.95 to 1:1.05;

According to another preferred aspect, when the coformer of the co-crystal is 2,4-dihydroxybenzoic acid, the molar ratio between Zanubrutinib and 2,4-dihydroxybenzoic acid is from 1:0.95 to 1:1.05.

According to another preferred aspect, when the coformer of the co-crystal is 4-aminobenzoic acid, the molar ratio of zanubrutinib to 4-aminobenzoic acid is from 1:0.95 to 1:1.05.

According to another preferred aspect, when the coformer of the co-crystal is 4-methylaminobenzoic acid, the molar ratio of zanubrutinib to 4-methylaminobenzoic acid is from 1:0.95 to 1:1.05.

According to another object, the invention therefore relates to a co-crystal of Zanubrutinib, wherein the coformer of the co-crystal is 3-hydroxybenzoic acid, the co-crystal being characterized by an XRPD diffractogram comprising a reflection at angle 2θ equal to 23.5°±0.2° or an onset peak in DSC at a temperature between 133.9° C. and 139.9° C., preferably between 134.9° C. and 138.9° C. Other features related to this specific co-crystal are as noted above.

According to another object, the invention thus relates to a co-crystal of Zanubrutinib, wherein the coformer of the co-crystal is 2,4-dihydroxybenzoic acid, the co-crystal being characterized by an XRPD diffractogram comprising a reflection at angle 2θ even at 21.1°±0.2° or an onset peak in DSC at a temperature between 138.3° C. and 144.3° C., preferably between 139.3° C. and 143.3° C. Other features related to this specific co-crystal are as noted above.

According to another object, the invention therefore relates to a co-crystal of Zanubrutinib, wherein the coformer of the co-crystal is 4-aminobenzoic acid, the co-crystal being characterized by an XRPD diffractogram comprising a reflection at angle 2θ equal to 23.6°±0.2° or an onset peak in DSC at a temperature between 141.5° C. and 147.5° C., preferably between 142.5° C. and 146.5° C. Other features related to this specific co-crystal are as noted above.

According to another aspect, the invention therefore relates to a co-crystal of Zanubrutinib, wherein the coformer of the co-crystal is 4-methylaminobenzoic acid, the co-crystal being characterized by an XRPD diffractogram comprising a reflection at angle 2θ equal to 22.8°±0.2° or an onset peak in DSC at a temperature between 136.9° C. and 142.9° C., preferably between 137.9° C. and 141.9° C. Other features related to this specific co-crystal are as noted above.

According to another object, the invention therefore relates to a process for preparing Zanubrutinib co-crystal comprising the steps of:
a) reacting a compound of formula (II) or a salt thereof:

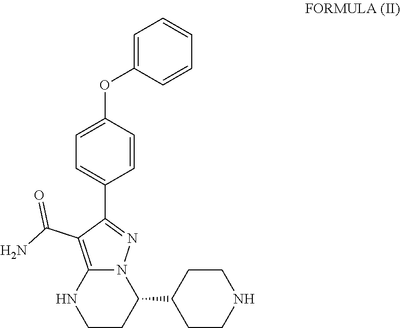 [SMILES: NC(=O)c1c(-c2ccc(Oc3ccccc3)cc2)nn2c1NCC[C@H]2C1CCNCC1]

with acryloyl chloride to form zanubrutinib;
b) adding a coformer selected from: 3-hydroxybenzoic acid, 2,4-dihydroxybenzoic acid, 4-aminobenzoic acid and 4-methylaminobenzoic acid, to obtain Zanubrutinib co-crystal.

The above is to be understood by way of example and not as a limitation. Furthermore, the person skilled in the art will be able to understand that modifications can be made without thereby departing from the scope of the present invention.

### EXAMPLES

### Example 1—Synthesis of Zanubrutinib Co-Crystal

2.84 g (4.79 mmol) of the compound of formula (II) salified with L-DBTA (L-dibenzoyltartaric) acid (2:1), acetonitrile (28 mL), and demineralized water (14 mL) were loaded into a flask. The suspension was left under stirring and NaOH 30% aq (1.5 eq, 0.958 g) was flush added. It was stirred again for 15 minutes. Ethyl acetate (17 ml) was loaded and the phases were separated. The organic phase was washed with 15% aq. NaCl and the final organic phase was concentrated to a residual 10 ml. 30 ml of acetonitrile were loaded and concentrated to a residual 24 ml. 25 ml of demineralized water, 1.2 g of NaHCO₃ (3 eq) and 358 mg of L-tartaric acid (0.5 eq) were then loaded into the flask. The temperature was brought to −2° C. and acryloyl chloride 564 mg (1.3 eq) was dropwise added. After 15 minutes isopropyl acetate (12 ml) was added and the phases were separated. The organic phase was washed with NaHCO₃ aq. at 5% and isopropyl acetate (40 ml) and the coformer selected from 3-hydroxybenzoic acid, 2,4-dihydroxybenzoic acid, 4-aminobenzoic acid and 4-methylaminobenzoic acid (1.1 eq) were loaded onto the organic phase. It was concentrated to a small volume and taken up again with more isopropyl acetate until the acetonitrile was removed. The final solution was heated to 40° C. and primed. When abundant precipitate was observed, it was filtered and washed with 2 ml of isopropyl acetate. The co-crystal was dried in an oven at 45° C. under vacuum for 6 hours.

### Example 2—Synthesis of Amorphous Zanubrutinib

2 g of Zanubrutinib cocrystal and 6 ml of DMSO were loaded into a flask. It was stirred until completely dissolved. A solution of 70 ml of NaHCO₃ 5% aq. was prepared in another flask and the DMSO solution was slowly dropped onto the aqueous one, maintaining vigorous stirring. The suspension was allowed to spin for 1 hour, then filtered and washed with water. The solid was discharged and dissolved in 6 mL of DMSO. A solution of 70 ml of NaHCO₃ 5% aq. was prepared in another flask and the DMSO solution was slowly dropped onto the aqueous one, maintaining vigorous stirring. The suspension was allowed to spin for 1 hour, then filtered and washed with water. The solid was discharged and washed with 2 slurry in water for 1 hour each. The final amorphous solid was dried in an oven at 35° C. for 16 h.

### Characterization Studies

The following characterization studies were conducted

### ¹H-NMR

The ¹H-NMR spectrum was acquired with a Varian Mercury 300 spectrometer by dissolving the sample in dimethyl sulfoxide. 64 FIDs were acquired with a relaxation time of 2 s at 25° C.

### IR Spectrum

Data for the FT-IR spectrum was collected with an Attenuated Total

Reflectance (ATR) Frontier IR spectrophotometer at resolutions of 4 cm⁻¹ over a range of 4000 to 650 cm⁻¹.

### XRPD

XRPD were acquired with Bruker AXS D8 ADVANCE instrumentation. The samples were analyzed as they are. The operating conditions used are shown below.

### Operating Conditions

| Protractor: | theta/2-theta vertical | |
|---|---|---|
| Protractor Radius: | 250 | mm |
| Generator: | 3 | KW |
| Copper anode: | 2.2 | KW |
| Motorized primary slit assembly | | |
| Axial slit | 2.5° | |
| Detector | Fast liner SSD 160 | |
| Motorized anti-diffusion screen | | |
| X-Ray Generator: | Current intensity 40 mA, | |
| | voltage 40 kV | |
| Primary optics: | Fixed illumination 25.0 mm | |
| Screen knife height: | fixed 0.40 mm | |
| Rotation: | on | |
| 2Theta range: | 4.0°-35.0° in 0.025° 2Theta | |
| | increment | |
| Time/steps: | 0.2 | sec |
| Temperature: | 25° | C. |

### DSC

| coformer | Operating conditions |
|---|---|
| 3-Hydroxybenzoic acid | Aluminum crucible, nitrogen |
| | atmosphere, 50° C. to 220° C., at |
| | 10° C./min |
| 2,4-dihydroxybenzoic acid | Aluminum crucible, nitrogen |
| | atmosphere, 50° C. to 220° C., at |
| | 10° C./min |
| 4-aminobenzoic acid | Aluminum crucible, nitrogen |
| | atmosphere, 36° C. to 220° C., at |
| | 10° C./min |
| 4-methylaminobenzoic acid | Aluminum crucible, nitrogen |
| | atmosphere, 36° C. to 220° C., at |
| | 10° C./min |

### TGA Extension

| coformer | Operating conditions |
|---|---|
| 3-Hydroxybenzoic acid | nitrogen atmosphere, 30° C. to |
| | 160° C., at 10° C./min |
| 2,4-dihydroxybenzoic acid | nitrogen atmosphere, 30° C. to |
| | 160° C., at 10° C./min |
| 4-aminobenzoic acid | nitrogen atmosphere, 30° C. to |
| | 160° C., at 10° C./min |
| 4-methylaminobenzoic acid | nitrogen atmosphere, 30° C. to |
| | 160° C., at 10° C./min |

No mass loss was observed for any of the coformers.

The characterization data collected (NMR, DSC, TGA, IR, XRPD) for the batches of Zanubrutinib co-crystals (3-hydroxybenzoic acid, 2,4-dihydroxybenzoic acid, 4-aminobenzoic acid and 4-methylaminobenzoic acid) are according to the proposed structures.

Their molar ratios are as follows:
The molar ratio of zanubrutinib to 3-hydroxybenzoic acid is 1:0.95.
The molar ratio of zanubrutinib to 2,4-dihydroxybenzoic acid is 1:1.35.
The molar ratio of zanubrutinib to 4-aminobenzoic acid is 1:1
The molar ratio of zanubrutinib to 4-methylaminobenzoic acid is 1:1.20.

### HPLC Data

TABLE 1
HPLC data crystallization tests in 10 vol of ACN

| | | Formula | | Formula | Formula | Formula | Formula |
|---|---|---|---|---|---|---|---|
| Trial | coformer | III | Zanubrutinib | IV | V | VI | VII |
| Form A | / | 0.3 | 97.64 | 0.34 | 0.38 | 0.13 | 0.94 |
| 1850/57 | 3-OH | | | | | | |
| A | C6H5COOH | 0.29 | 98.83 | 0.06 | 0.41 | 0.11 | 0.18 |
| 1850/57 | 2,4-diOH | | | | | | |
| B | C6H5COOH | 0.25 | 99.18 | na | 0.21 | 0.07 | 0.1 |
| 1850/57 | 4-NH2 | | | | | | |
| C | C6H5COOH | 0.28 | 99.21 | na | 0.32 | 0.05 | 0.05 |
| 1850/57 | 4-MeNH | | | | | | |
| D | C6H5COOH | 0.29 | 99.17 | na | 0.41 | na | 0.04 |

HPLC data crystallization tests in 5 vol AcOEt

| | | Formula | | Formula | Formula | Formula | Formula |
|---|---|---|---|---|---|---|---|
| Trial | coformer | VIII | Zanubrutinib | V | VI | IX | VIII |
| Raw | / | 0.86 | 97.32 | 0.63 | 0.09 | 0.1 | 0.7 |
| 1850/57 | 3-OH | | | | | | |
| E | C6H5COOH | 0.18 | 98.92 | 0.47 | 0.07 | na | 0.2 |
| 1850/57 | 2,4-diOH | | | | | | |
| F | C6H5COOH | na | 99.48 | 0.28 | na | na | na |
| 1850/57 | 4-NH2 | | | | | | |
| G | C6H5COOH | 0.11 | 99.3 | 0.45 | na | na | 0.09 |
| 1850/57 | 4-MeNH | | | | | | |
| H | C6H5COOH | 0.13 | 99.21 | 0.41 | na | na | 0.16 |

The formulas of the III-IX impurities indicated in Table 1 are reported in the above description.

1. A process for preparing Zanubrutinib of formula (I) 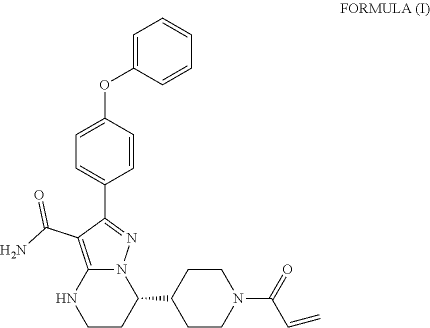 [SMILES: C=CC(=O)N1CCC([C@@H]2CCNc3c(C(N)=O)c(-c4ccc(Oc5ccccc5)cc4)nn32)CC1]
in amorphous form, comprising:
a) reacting a compound of formula (II) or a salt thereof: 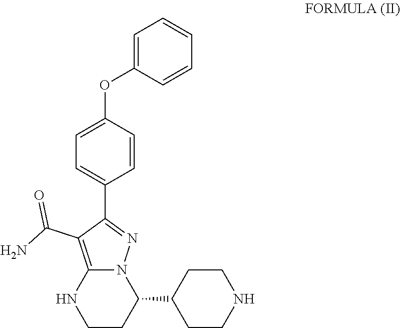 [SMILES: NC(=O)c1c(-c2ccc(Oc3ccccc3)cc2)nn2c1NCC[C@H]2C1CCNCC1]
with acryloyl chloride to form Zanubrutinib;
b) adding to the Zanubrutinib obtained in a) a coformer selected from the group consisting of: 3-hydroxybenzoic acid, 2,4-dihydroxybenzoic acid, 4-aminobenzoic acid and 4-methylaminobenzoic acid, to obtain a Zanubrutinib co-crystal; and
c) converting the Zanubrutinib co-crystal obtained in b) into Zanubrutinib in amorphous form by treatment of the co-crystal in basic pH water.

2. The process according to claim 1, wherein a) comprises reacting a salt of the compound of formula (II) with acryloyl chloride, wherein the salt of the compound of formula (II) is a salt of the compound of formula (II) with an acid selected from the group consisting of L-DBTA (L-dibenzoyltartaric acid), D-DBTA (D-dibenzoyltartaric acid), L-DTTA (Di-p-toluoyl-L-tartaric acid), D-DTTA (Di-p-toluoyl-D-tartaric acid), L-malic acid, D-malic acid, L-mandelic acid, D-mandelic acid, L-camphorsulfonic acid, D-camphorsulfonic acid, L-tartaric acid, and D-tartaric acid.

3. The process according to claim 1, wherein a) comprises reacting a salt of the compound of formula (II) with acryloyl chloride under basic conditions.

4. The process according to claim 1, wherein
when the coformer of the co-crystal is 3-hydroxybenzoic acid, the co-crystal is characterized by an XRPD diffractogram comprising a reflection at 2θ angle of 23.5°±0.2° or a DSC onset peak at a temperature between 133.9° C. and 139.9° C.;
when the coformer of the co-crystal is 2,4-dihydroxybenzoic acid, the co-crystal is characterized by an XRPD diffractogram comprising a reflection at 2θ angle of 21.1°±0.2° or a DSC onset peak at a temperature between 138.3° C. and 144.3° C.;
when the coformer of the co-crystal is 4-aminobenzoic acid, the co-crystal is characterized by an XRPD diffractogram comprising a reflection at 2θ angle of 23.6°±0.2° or a DSC onset peak at a temperature between 141.5° C. and 147.5° C.; or
when the coformer of the co-crystal is 4-methylaminobenzoic acid, the co-crystal is characterized by an XRPD diffractogram comprising a reflection at 2θ angle of 22.8°±0.2° or a DSC onset peak at a temperature between 136.9° C. and 142.9° C.

5. The process according to claim 1, wherein in the Zanubrutinib co-crystal a molar ratio between Zanubrutinib and coformer is in a range of 1:0.95 to 1:1.05.

6. A Zanubrutinib co-crystal, wherein a co-crystal coformer is selected from the group consisting of: 3-hydroxybenzoic acid, 2,4-dihydroxybenzoic acid, 4-aminobenzoic acid and 4-methylaminobenzoic acid;
wherein:
when the coformer of the co-crystal is 3-hydroxybenzoic acid, the co-crystal is characterized by an XRPD diffractogram comprising a reflection at 2θ angle of 23.5°±0.2° or a DSC onset peak at a temperature between 133.9° C. and 139.9° C.;
when the coformer of the co-crystal is 2,4-dihydroxybenzoic acid, the co-crystal is characterized by an XRPD diffractogram comprising a reflection at 2θ angle of 21.1°±0.2° or a DSC onset peak at a temperature between 138.3° C. and 144.3° C.;
when the coformer of the co-crystal is 4-aminobenzoic acid, the co-crystal is characterized by an XRPD diffractogram comprising a reflection at 2θ angle of 23.6°±0.2° or a DSC onset peak at a temperature between 141.5° C. and 147.5° C.; or
when the coformer of the co-crystal is 4-methylaminobenzoic acid, the co-crystal is characterized by an XRPD diffractogram comprising a reflection at 2θ angle of 22.8°±0.2° or a DSC onset peak at a temperature between 136.9° C. and 142.9° C.

7-9. (canceled)

10. A process for preparing a Zanubrutinib co-crystal comprising:
a) reacting a compound of formula (II) or a salt thereof: 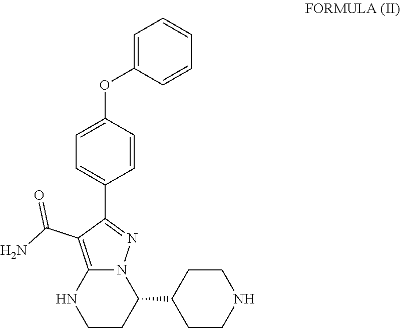 [SMILES: NC(=O)c1c(-c2ccc(Oc3ccccc3)cc2)nn2c1NCC[C@H]2C1CCNCC1]
with acryloyl chloride to form Zanubrutinib; and
b) adding to the Zanubrutinib obtained in a) a coformer selected from the group consisting of: 3-hydroxybenzoic acid, 2,4-dihydroxybenzoic acid, 4-aminobenzoic acid and 4-methylaminobenzoic acid, to obtain a Zanubrutinib co-crystal.

11. The Zanubrutinib co-crystal of claim 6, wherein the coformer is 3-hydroxybenzoic acid.

12. The Zanubrutinib co-crystal of claim 6, wherein the coformer is 2,4-dihydroxybenzoic acid.

13. The Zanubrutinib co-crystal of claim 6, wherein the coformer is 4-aminobenzoic acid.

14. The Zanubrutinib co-crystal of claim 6, wherein the coformer is 4-methylaminobenzoic acid.
